(19) United States
(12) Patent Application Publication
(10) Pub. No.: US 2004/0067214 A1
(43) Pub. Date: Apr. 8, 2004

(54) Odor control system

(75) Inventors: Patsy A. Krautkramer, Omro, WI (US); William G. Reeves, Appleton, WI (US); Heather A. Sorebo, Appleton, WI (US); Emmanuelle C. Damay, Erlangen (DE)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/367,410

(22) Filed: Feb. 13, 2003

Related U.S. Application Data
(63) Continuation-in-part of application No. 10/267,376, filed on Oct. 8, 2002, now abandoned.

(51) Int. Cl. A61L 9/01; A61L 9/04

(52) U.S. Cl. 424/76.3

(57) ABSTRACT

A method and system (22) for reducing malodor includes a providing of an operative quantity of particles of adsorbent material with a carrier structure (24), and a configuring of the carrier structure to contact at least one viscous, aqueous body-liquid during an intended use of the carrier structure. The carrier structure (24) has been configured to reduce malodor from the viscous body-liquid. In particular features, particles of adsorbent material (28) have been configured to include a total quantity of particle pores, and at least a significant portion of the particle pores have been configured to provide an operative quantity of target pores. In other features, the target pores have been configured to withdraw and hold water from said body-liquid, and have been configured to hold the water in a manner that renders the water substantially inaccessible to odor-causing organisms. In a further feature, the target pores can be configured to provide a selected target pore-size. In still another feature, the particles of adsorbent material can be configured to carry an operative quantity of odor-treatment material.

### CROSS-REFERENCE TO RELATED APPLICATIONS:

This is a continuation-in-part of U.S. patent application Ser. No. 10/267,376 entitled ODOR CONTROL SYSTEM by P. Krautkramer et al. which was filed Oct. 8, 2002, the entire disclosure of which is incorporated herein by reference in a manner that is consistent herewith.

### BACKGROUND

The present invention relates to a system and method for reducing malodor in a selected article. In particular aspects, the present invention relates to a system and method that can distinctively employ adsorbent particles to reduce the occurrence of malodor.

The use of adsorbent particles in disposable absorbent articles is known. Such adsorbent particles are generally employed on a somewhat limited basis for odor control in disposable personal care absorbent articles. The conventional adsorbent particles have been configured to gather and hold the malodorous compounds, and a more widespread use of such adsorbent particles in absorbent structures and disposable absorbent articles has been somewhat confined by the limited efficacy of the adsorbent particles in the handling of complex liquids. As a result, there has been a continued need for improved systems and methods that can more effectively control malodor in the presence of liquid-complexes, such as complex body-liquids. Additionally, such improved systems and methods can potentially enhance the operation of the adsorbent particles in disposable absorbent articles, as well as other absorbent structures.

### BRIEF DESCRIPTION OF THE INVENTION

The present inventors have recognized the difficulties and problems that are present in the prior art, and in response thereto, have conducted intensive research into developing adsorbent particles that can more effectively handle and process complex body-liquids. While conducting such research, the inventors found that certain adsorbent materials exhibit an enhanced efficacy in the handling of malodor arising from complex liquids. The efficacy of these adsorbent materials can be improved by an appropriate selection and configuration of the adsorbent materials. In a particular aspect, the adsorbent materials can provide an improved ability to reduce the activity of mcro-organisms that help generate malodor. As a result of the improved performance, the adsorbent materials of the present invention can allow for a distinctive incorporation and use of adsorbent particles in disposable absorbent articles, disposable personal care articles, and other absorbent structures.

Generally stated, the present invention can provide a method for reducing malodor, which includes a providing of an operative quantity of particles of adsorbent material with a carrier structure, and a configuring of the carrier structure to contact at least one viscous, aqueous body-liquid during an intended use of the carrier structure. In a particular feature, the particles of adsorbent material have been configured to include a total quantity of particle pores, and at least a significant portion of the particle pores have been configured to provide an operative quantity of target pores. In other features, the target pores have been configured to withdraw and hold water from said body-liquid, and have been configured to hold the water in a manner that renders the water substantially inaccessible to odor-causing organisms.

The present invention can also provide a system for reducing malodor in a personal care article. The personal care article configured to retain at least one viscous, aqueous body-liquid, and the system can include at least one adsorbent particulate material disposed within the personal care article. The at least one adsorbent particulate material can be configured to provide a total quantity of particle pores configured to withdraw and hold water from at least one viscous, aqueous body-liquid. A substantial portion of the total quantity of particle pores can be configured to hold water in a manner that renders the water substantially inaccessible to odor-causing organisms. An operative quantity of such adsorbent particulate material can be disposed within the personal care article to operatively contact the at least one viscous, aqueous body-liquid during use such that water from the viscous, aqueous body-fluid is rendered substantially inaccessible to odor-causing organisms, to thereby reduce malodor from the personal care article.

In another aspect, a method for reducing malodor comprises a providing of an operative quantity of particles of adsorbent material with a carrier structure, and a configuring of the carrier structure to reduce malodor from at least one viscous, aqueous body-liquid. The particles of adsorbent material have been configured to include a total quantity of particle pores. The particles of adsorbent material can be configured to include an operative quantity of odor-treatment material, and the odor-treatment material can be configured to cooperate with the viscous body-liquid to thereby generate a substantially non-objectionable odor.

In a particular feature, the target pores can be configured to provide a selected target pore-size. In another feature, the particles of adsorbent material can be configured to carry an operative quantity of a selected odor-treatment material.

In its various aspects and features, the present invention can provide a method and system that can more effectively reduce malodor in a personal care article. In particular, the present invention can provide a method and system that can more effectively reduce malodor in a personal care article that has been configured to hold liquid-complexes, such as complex body-liquids. As a result, the article can have a longer period of use, and can better maintain a discreet condition. Additionally, the article can better retain a condition that is comfortable and non-obtrusive.

### DRAWINGS

The various features, aspects and advantages of the present invention will become better understood with reference to the following description, appended claims and accompanying drawings where:

FIG. 1 shows a representative tampon article which incorporates the odor-control system of the invention.

FIG. 2 shows a representative end-wise view of a transverse cross-section through a tampon article which incorporates the system of the invention.

FIG. 2A shows a representative end-wise view of a transverse cross-section through another tampon article which incorporates the system of the invention.

FIG. 3 representatively shows a partially cut-away, top plan view of a pad article which incorporates the system of the invention and has a bodyside layer and a garment-side layer.

FIG. 4 shows a representative end-wise view of a cross-section through a pad article which incorporates a bodyside layer and a garment-side layer.

FIG. 5 is a schematic end-view of a cross-section through a representative absorbent article having an odor-control system and an absorbent retention portion.

FIG. 6 is a schematic end-view of a cross-section through a representative absorbent article having another arrangement of an odor-control system and a retention portion.

FIG. 7 is perspective view of an absorbent article having an odor-control system and a retention portion.

FIG. 8 is a top, plan view of a representative absorbent article having an odor-control system and wing members for holding the article in an undergarment.

FIG. 9 is a schematic end-view of a cross-section through a representative absorbent article having an odor-control system which is partially sunken into a thickness of a retention portion.

FIG. 9A is a schematic end-view of a cross-section through a representative absorbent article having an odor-control system which is sunken through a total thickness of a retention portion.

FIG. 9B is a schematic end-view of a cross-section through a representative absorbent article having an odor-control system which is superposed onto a retention portion.

FIG. 10 is a schematic end-view of a cross-section through a representative absorbent article having an odor-control system which is incorporated into two or more layer regions.

FIG. 10A is a schematic end-view of a cross-section through a representative absorbent article having an odor-control system which is incorporated into one or more edge layer regions.

FIG. 11 is a perspective view of a cross-section through a representative article having an odor-control system arranged in a medial position with respect to an array of absorbent components that are distributed along the x-y, width and length dimensions of the article.

FIG. 11A is a perspective view of a cross-section through another representative article having an odor-control system arranged in an intermediate position with respect to an array of absorbent components that are distributed along x-y dimensions of the article.

FIG. 11B is a perspective view of a cross-section through still another representative article having an odor-control system arranged in an outboard position with respect to an array of absorbent components that are distributed along x-y dimensions of the article.

FIG. 12 is a representative top, plan view of an article having an odor-control system that is configured with an array of individual odor-control components that are distributed along x-y dimensions of the article.

FIG. 13 is a representative, perspective view of a partially sectioned article having an odor-control system configured with a varying, depth-wise contour.

### DETAILED DESCRIPTION

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The adsorbent materials employed with the present invention can include any operative adsorbent particles, alone or in combination with other treatments or additives. For example, the adsorbent particles can include adsorbent particles that are treated with a surface modifying agent. By the terms “particle,” “particles,” “particulate,” “particulates” and the like, it is meant that the adsorbent material is generally in the form of discrete units. The units can comprise granules, powders, spheres, pulverized materials or the like, as well as combinations thereof. The particles can have any desired shape such as, for example, cubic, rod-like, polyhedral, spherical or semi-spherical, rounded or semi-rounded, angular, irregular, etc. Shapes having a large greatest dimension/smallest dimension ratio, like needles, flakes and fibers, are also contemplated for inclusion herein. The terms “particle” or “particulate” may also include an agglomeration comprising more than one individual particle, particulate or the like. Additionally, a particle, particulate or any desired agglomeration thereof may be composed of more than one type of material.

As used herein, the term “nonwoven” refers to a fabric web that has a structure of individual fibers or filaments which are interlaid, but not in an identifiable repeating manner.

As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the phrase “meltblown fibers” refers to fibers formed by extruding a molten thermoplastic material through a plurality of fine, usually circular, die capillaries as molten threads or filaments into a high velocity, usually heated, gas (e.g., air) stream which attenuates the filaments of molten thermoplastic material to reduce their diameter. Thereafter, the meltblown fibers are carried by the high velocity gas stream and are deposited on a collecting surface to form a web of randomly disbursed meltblown fibers.

“Coform” as used herein is intended to describe a blend of meltblown fibers and cellulose fibers that is formed by air forming a meltblown polymer material while simultaneously blowing air-suspended cellulose fibers into the stream of meltblown fibers. The meltblown fibers containing wood fibers are collected on a forming surface, such as provided by a foraminous belt. The forming surface may include a gas-pervious material, such as spunbonded fabric material, that has been placed onto the forming surface.

As used herein, the phrase “complex liquid” describes a liquid generally characterized as being a viscoelastic liquid comprising multiple components having inhomogeneous physical and/or chemical properties. It is the inhomogeneous properties of the multiple components that challenge the efficacy of an adsorbent material in the handling of complex liquids. In contrast with complex liquids, simple liquids, such as, for example, urine, physiological saline, water and the like, are generally characterized as being relatively low-viscosity and comprising one or more components having homogeneous physical and/or chemical properties. As a result of having homogeneous properties, the one or more components of simple liquids behave substantially similarly during absorption or adsorption.

Although a complex liquid is generally characterized herein as including specific components having inhomogeneous properties, each specific component of a complex liquid generally has homogeneous properties. Consider for example a representative complex body-liquid having three specific components: red blood cells, blood protein molecules and water molecules. Upon examination, one skilled in the art could easily distinguish between each of the three specific components according to their generally inhomogeneous properties. Moreover, when examining a particular specific component such as the red blood cell component, one skilled in the art could easily recognize the generally homogeneous properties of the red blood cells.

As used herein, the phrase “absorbent article” refers to devices which absorb and contain body liquids, and more specifically, refers to devices which are placed against or near the skin to absorb and contain the various liquids discharged from the body. The term “disposable” is used herein to describe absorbent articles that are not intended to be laundered or otherwise restored or reused as an absorbent article after a single use. Examples of such disposable absorbent articles include, but are not limited to: health care related products including surgical drapes, gowns, and sterile wraps; personal care absorbent products such as feminine hygiene products (e.g., sanitary napkins, pantiliners, interlabial devices and the like), infant diapers, children's training pants, adult incontinence products and the like; as well as absorbent wipes and covering mats.

Disposable absorbent articles such as, for example, many of the personal care absorbent products, can include a liquid pervious topsheet, a substantially liquid impervious backsheet joined to the topsheet, and an absorbent core positioned and held between the topsheet and the backsheet. The topsheet is operatively permeable to the liquids that are intended to be absorbed by the absorbent article, and the backsheet is substantially impermeable or otherwise operatively impermeable to the intended liquids. The absorbent articles may also include other components, such as liquid wicking layers, liquid distribution layers, barrier layers, and the like, as well as combinations thereof. Disposable absorbent articles and the components thereof, can operate to provide a body-facing surface and a garment-facing surface. As used herein, “body-facing surface” means that surface of the article or component which is intended to be disposed toward or placed adjacent to the body of the wearer, while the “garment-facing surface” is on the opposite side, and is intended to be disposed toward or placed adjacent to the wearer's undergarments when the disposable absorbent article is worn.

With reference to FIGS. 1 through 4, the present invention can provide an odor-control method for reducing malodor, which includes a providing of an operative quantity of particles of adsorbent material 28 with a carrier structure or mechanism, such as provided by a structure which includes a carrier layer 24. The carrier layer is desirably flexible, but may be relatively inflexible, as desired. The carrier structure can be configured to contact at least one viscous, aqueous body-liquid during an intended use of the carrier structure. Additionally, the carrier structure can be distinctively configured to reduce malodor arising from the presence of viscous body-liquid. In a particular feature, the particles of adsorbent material can be configured to include a total quantity of particle pores, and at least a significant portion of the particle pores can be configured to provide an operative quantity of target pores. In other features, the target pores can be configured to withdraw and hold water from said body-liquid, and can be configured hold the water in a manner that renders the water substantially inaccessible to odor-causing organisms. It should be readily appreciated that the held water may contain dissolved materials, such as ions, dissolved proteins or other dissolved compounds, as well as combinations thereof.

The present invention can also provide a distinctive odor-control system 22 for reducing malodor in a personal care article 20. The personal care article configured to retain at least one viscous, aqueous body-liquid, and the odor-control system can be configured to include at least one adsorbent particulate material 28 disposed within the personal care article. The at least one adsorbent particulate material can be configured to provide a total quantity of particle pores configured to withdraw and hold water from the at least one viscous, aqueous body-liquid. A substantial portion of the total quantity of particle pores can be configured to hold water in a manner that renders the water substantially inaccessible to odor-causing organisms. An operative quantity of such adsorbent particulate material can be disposed within the personal care article to operatively contact the at least one viscous, aqueous body-liquid during use such that water from the viscous, aqueous body-fluid is rendered substantially inaccessible to odor-causing organisms, thereby reducing malodor from the personal care article.

In a further aspect, a method and system for reducing malodor can include a providing of an operative quantity of particles of adsorbent material 28 with a carrier structure, and a configuring of the carrier structure to reduce malodor from at least one viscous, aqueous body-liquid. The particles of adsorbent material can be configured to include a total quantity of particle pores, and the particles of adsorbent material can be configured to incorporate or otherwise include an operative quantity of odor-treatment material. Additionally, the odor-treatment material can be configured to cooperate with the viscous body-liquid to thereby generate a substantially non-objectionable odor or fragrance.

In a particular feature, the target pores can be configured to provide a selected target pore-size and/or pore-size distribution. In another feature, target pores can constitute a selected percentage of the total quantity of pores provided by the adsorbent material.

In its various aspects and features, alone or in combination, the present invention can provide a method and system that can more effectively reduce malodor in a personal care article, such as an absorbent, personal care article. In a particular aspect, the present invention can provide a method and system that can more effectively reduce malodor in a personal care article that has been configured to hold liquid-complexes, such as complex body-liquids. The method and system can inhibit the proliferation of undesired malodor-causing organisms while substantially maintaining any desired, “friendly” bacteria or other “friendly” organisms in the body-environment. The article can have a longer period of use, and can better maintain a discreet condition. Additionally, the article can better retain a condition that is comfortable and non-obtrusive. As a result, the method and system can help provide improved confidence to the wearer, and can help provide more economical use.

The odor-control particulate materials employed with the present invention can be appropriately combined with an operative carrier system or structure to provide the desired odor-control system 22. In a particular aspect, the particulate material 28 can be held or otherwise carried by or within a suitable containment system or mechanism. Any system or mechanism which is capable of holding or otherwise carrying the selected adsorbent materials, and is capable of being operatively located in a disposable absorbent article, can be employed in the present invention. Many such carrier or containment systems or mechanisms are known to one skilled in the art. For example, the carrier structure may include a fibrous matrix such as an airlaid or wet laid web of cellulosic fibers, a meltblown web of synthetic polymeric fibers, a spunbonded web of synthetic polymeric fibers, a coformed matrix comprising cellulosic fibers and fibers formed from a synthetic polymeric material, airlaid heat-fused webs of synthetic polymeric material, open-celled foams, or the like, as well as combinations thereof.

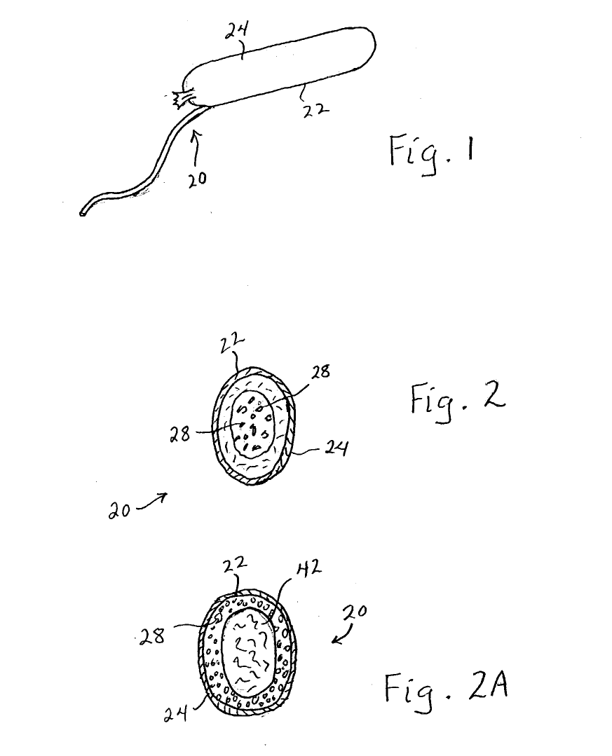

The odor-control system 22 can include at least one flexible carrier layer 24, and the carrier layer can be provided by any operative material. For example, the carrier layer may be an individual material or a composite material. The carrier layer 24 is sufficiently flexible to provide comfort and conformability, and can be configured to help direct bodily exudates away from the body of the wearer and toward a selected retention portion 42 of the article (e.g. FIGS. 2A, 5, 6). In a desired feature, the carrier layer 24 can be configured to retain little or no liquid in its structure, and may be configured and arranged to provide a relatively comfortable and non-irritating surface next to the body tissues of a female wearer. The carrier layer may be generally liquid-impermeable, but desirably is operatively liquid-permeable. More particularly, the carrier layer is permeable to water. Accordingly, at least the aqueous portion of a complex-liquid can pass through the carrier layer. The carrier layer 24 can be constructed of any material which is also easily penetrated by bodily fluids contacting its surface. For example, the carrier layer can include a woven fabric, a nonwoven fabric, a polymeric film that has been configured to be operatively liquid-permeable, or the like, as well as combinations thereof. Examples of suitable materials for constructing the carrier layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A particular example of a suitable carrier layer material can include a bonded-carded-web composed of polypropylene and polyethylene. Other examples of suitable materials are composite materials of a polymer and a nonwoven fabric material. The composite materials are typically in the form of integral sheets generally formed by the extrusion of a polymer onto a web of spunbond material. The liquid-permeable carrier layer 24 may optionally contain a plurality of apertures (not shown) formed therein which can increase the rate at which bodily liquids can move through the thickness of the carrier layer.

The carrier layer 24 may also include a physiologically hydrous material. As used herein, the term “physiologically hydrous” is intended to connote a cover material which can maintain a suitably moist interface between the absorbent article 20 and any contacting body-tissues of the wearer that are ordinarily moist. For example, such moist-tissue regions are present in the vulvovaginal area of the female anatomy. The physiologically hydrous cover material can provide a desired level of comfort when disposed within the selected, moist-tissue environment of the wearer. Thus, while the carrier layer may not be “hydrous” in the classic sense, inasmuch as the carrier layer will be substantially dry prior to use on the wearer, the carrier layer 24 can maintain, or at least can avoid excessive interference with, a hydration level or balance that is desired within the ordinarily-moist body tissue.

Physiologically hydrous materials are, for example, described in detail in U.S. Pat. No. 4,846,824 to F. Lassen et al., and in U.S. Pat. No. 5,891,126 to Osborn III et al. The entire disclosures of these documents are incorporated herein by reference in a manner that is consistent herewith.

The carrier layer 24 can also have at least a portion of its bodyside surface treated with a surfactant to render the carrier layer more hydrophilic. The surfactant can permit arriving bodily liquids to more readily penetrate the carrier layer. The surfactant may also diminish the likelihood that the arriving bodily liquids, such as menstrual fluid, will flow off the carrier layer rather than penetrate through the carrier layer. In a particular configuration, the surfactant can be substantially evenly distributed across at least a portion of the upper, bodyside surface of the carrier layer 24 that overlays an upper, bodyside surface of a selected retention portion of the article.

The carrier layer 24 may be maintained in secured relation with other components of the article by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the selected component with portions of the adjacent surface of the carrier layer, or fusing at least portions of the adjacent surface of the carrier layer to portions of the adjacent surface of the selected component.

The carrier layer 24 can typically be positioned at or operatively near at least one body-contacting surface of the article. Additionally, the carrier layer can be configured to partially or entirely surround and enclose the odor-control particulate material 28. The odor-control particulate material employed in the article or system of the invention can include any operative particulate material. In desired arrangements, the odor-control particulate material can include particles of an adsorbent material, and the particulate adsorbent material can be configured to include one or more distinctive parameters. While a wide variety of adsorbent materials are known, the present invention can incorporate a distinctive selection of the adsorbent materials that are suitable for use in the handling of complex liquids such as, for example, blood, menses, loose feces, vaginal discharges, nasal discharges and the like, as well as combinations thereof. Adsorbent materials suitable for use in the handling of complex liquids desirably are substantially wettable or hydrophilic with respect to the complex liquids, thus allowing the complex liquids to spread over the surface of the adsorbent materials. In addition, the adsorbent materials employed with the present invention are desirably in particle form and substantially insoluble in the complex liquids. It is further desired that the adsorbent materials of the present invention be substantially inert, and neither substantially soften nor substantially swell during adsorption. Any such suitable adsorbent material desirably has a high surface area relative to its weight, as determined by an appropriate measuring method such as gas adsorption, cetyltrimethyl ammonium bromide adsorption or mercury intrusion porosimetry. Suitable methods are described in detail in ASTM D2414-00, “Standard Test Method for Carbon Black—n-Dibutyl Phthalate Absorption Number”, published March 2000; in ASTM D3765-99, “Standard Test Method for Carbon Black—CTAB (Cetyltrimethylammonium Bromide) Surface Area”, published September 1999; and in Analytical Methods in Fine Particle Technology, authored by Paul A. Webb and Clyde Orr, and published by Micromeritics Instrument Corporation, Norcross, Ga.

Adsorbent materials suitable for use in the present invention include, but are not limited to, organic materials, inorganic materials and combinations thereof. Suitable inorganic materials include, for example, activated carbon, silicates, metal oxides, zeolites, carbonates, phosphates, borates, aerogels and combinations thereof. Suitable organic materials include, for example, cellulosic materials, starches, chitins, alginates, synthetic polymers or the like, as well as combinations thereof.

The adsorbent material may optionally be treated with a surfactant or other surface-modifying agent prior to incorporation into any containment means. Many materials are useful in this application, for example sulfonated alkyl and aryl compounds, ethoxylated alcohols and amines, polyamides and their derivatives, polysaccharides and their derivatives, polyethylene glycols and their derivatives, betaines and other zwitterionic compounds, and silyl compounds, as well as combinations thereof. Appropriate methods and techniques for incorporating the adsorbent material into the desired article are well known to one skilled in the art.

When used in a feminine hygiene product, the adsorbent material of the present invention can have a certain desirable distribution of pore sizes. In a bed of adsorbent particles, the pores can be provided by the spaces between particles (interstitial spaces), as well as an internal pore structure of the particles themselves. These interstitial spaces are linked to form what can be considered as a network of interstitial spaces. When a liquid moves into or through a bed of particles, the liquid generally moves through these interstitial spaces. These interstitial spaces that the liquid moves through can also be referred to as interstitial pores.

Since the walls of an interstitial pore are the surfaces of the particles themselves, the shape and size of the interstitial pores are usually determined by the particles themselves. Varying the size of the particles by varying their average dimensions or the distribution of their dimensions, varies the shape and size of the interstitial pores. Interstitial pores play a significant role in the intake rate and retention of a complex liquid by adsorbent particles.

Adsorbent materials suitable for use desirably have an acceptable rate of intake with respect to complex liquids. This acceptable rate of intake can be achieved through a heterogeneous distribution of pore sizes, and as previously discussed, a combination of particle sizes can provide an appropriately heterogeneous distribution of pore sizes. The pore sizes can suitably range from about 1,000 microns (micrometer) to about 0.2 microns (μm), where pore sizes between about 1,000 μm to about 100 μm can be primarily useful for the rapid intake and distribution of a complex liquid, and pore sizes between about 100 μm and about 0.2 μm can be primarily useful for the separation and retention of the components of a complex liquid.

The pore size distribution may be measured by the Capillary Tension Test identified in the TESTING section of the present disclosure, or by employing mercury porosimetry. Additionally, the pore size distribution can be indirectly measured by the Gel Bed Permeability Test identified in the TESTING section of the present disclosure. Mercury porosimetry can be conducted by a commercially available testing laboratory, such as Micromeritics, a business having offices located in Norcross, Ga., U.S.A., or Quantachrome, a business having offices located in Syosset, New York, U.S.A. For example, mercury porosimetry data regarding pore size, pore volume and pore size distribution can be obtained from Micromeritics Instrument Corp., One Micromeritics Dr., Norcross, Ga. 30093 U.S.A. The testing can include Macro and Meso Volume/Size Distribution by Mercury Intrusion Porosimetry, Test No. 005-65000-31, and test samples can be run on Micromeritics Instrument Corp.'s AUTOPORE Mercury Porosimeter, Unit 750.

Adsorbent particles are capable of retaining liquid in the interstitial pores or spaces between the particles as well as in the internal pores of the individual particles. It is desirable that the pores of an individual particle are accessible from the surface of the particle to adsorb the liquid. Liquid is capable of entering the internal pore volume of an individual particle through capillary forces. The addition of internal pores allows the liquid or liquid portion of the complex liquid to be retained by capillary force within the internal pores. This creates a dry feeling against the body, and diminishes the amount of free liquid in the bed of adsorbent particles. Consequently, the adsorbent particles can help minimize rewet. Suitable adsorbents can have a range of internal pore sizes from about 100 μm to about 0.2 μm to adsorb different sized components of a complex liquid and thus minimize liquid rewet as measured by the Rewet and Centrifuge Retention test methods described herein.

Based on the foregoing, adsorbent materials suitable for use in the present invention have the following parameters: wettable, stable when exposed to aqueous liquid, suitable interstitial pore size distribution for acceptable intake rate, and suitable internal pore size distribution for desired retention. Additionally, the adsorbent materials can have a suitable pore size distribution for a desired control of malodor. In a particular feature, the adsorbent materials can have a suitable internal pore size distribution that is configured to provide the desired control of malodor.

In the various arrangements of the present invention, other parameters of the adsorbent material may be desirable. For instance, when the complex liquid is menses and the adsorbent material is used in feminine hygiene products, the adsorbent materials employed with the present invention can have a particle size of between about 1,000 to about 100 microns (μm); and, more desirably, between about 850 to about 150 microns. It has been found that particles of adsorbent material having a size above about 1,000 microns are generally, readily perceptible to the wearer of any containment or carrier structure that supports the adsorbent materials of the present invention. It has also been found that particles of adsorbent material having a size below 100 microns are difficult to contain within any containment mechanism that readily allows complex liquids to penetrate through the containment mechanism to the adsorbent materials. It should be understood that the particles of adsorbent material falling within the range identified herein may comprise individual porous particles, or may be agglomerated particles which each agglomerated particle comprising a plurality of smaller particles composed of one or more types of adsorbent materials.

Another desirable specific parameter is retention capacity, which is expressed as the weight, (e.g. in grams) of the liquid retained, divided by the weight (e.g. in grams) of the adsorbent employed. For instance, where the complex liquid is menses and the adsorbent material is incorporated into a feminine hygiene product, the capacity can be determined by employing the Retention Capacity test identified in the present disclosure. The complex-liquid retention capacity of the adsorbent can be between about 1 and about 15 g/g; alternatively, between about 2 and about 8 g/g; and finally, alternatively, between about 2 and about 6 g/g. It is believed that adsorbent materials having lower retention capacities than 2 g/g would require the use of such large amounts of adsorbent material that users may find the feminine hygiene product to be excessively heavy. The complex liquid retention capacity can be estimated by summing the amount of pore volume between about 100 and about 0.2 microns in diameter. The pore volume can be determined, for instance, by capillary tension or mercury intrusion porosimetry. The complex liquid retention capacity can be limited by the strength of the pore wall material.

As previously mentioned, a mixture of particle sizes can be desirable to improve liquid intake and retention. Sufficient interstitial pores between particulates may be desired so that menses can rapidly enter into the bed of adsorbent particles and be distributed between the particulates. This property may be controlled with the particle size distribution of the adsorbent material. Generally, a broad particle size distribution may be desired to improve liquid intake and retention. A broad particle size distribution is used herein to describe a distribution having a standard deviation greater than 25 percent of the mean value.

If it is desired to move the liquid into and through the bed of particles relatively quickly, it can be advantageous to minimize the variation in the interstitial pore size and shape along the length of the interstitial pore. Consequently, a relatively wide distribution of particle sizes would create interstitial pores that allow the liquid to move into and through the bed of particles relatively quickly. Should the variation in the size and the packing of the particles become too large, such that some of the particles actually move within the interstitial pores themselves, the movement of liquid within and through the bed may not be relatively quick and may, instead, be relatively slow.

The inventors have also found that a combination of pore sizes can be effective at adsorbing a complex liquid. A bi-modal or multi-modal particle size distribution can be particularly desirable for producing a combination of pore sizes that can be desirable for enhancing the intake and retention of a complex liquid. One manner of achieving a desired pore size distribution can be to combine adsorbent particles of various sizes.

Where the adsorbent or other particulate material is configured for use in a method or system for reducing malodor, the method or system can include a providing of an operative quantity of particles of odor-control material with a carrier structure. The carrier structure has been configured to contact at least one viscous, aqueous body-liquid during an intended use of the carrier structure, and to thereby provide a desired reduction of malodor arising from the viscous body-liquid. The particles of adsorbent or other odor-control material have been configured to include a total quantity of particle pores, and at least a significant portion of the particle pores have been configured to provide an operative quantity of target pores. The target pores have been configured to withdraw and hold water from the body-liquid, and the target pores having been configured to hold the water in a manner that renders the water substantially inaccessible to odor-causing organisms. Desirably the target pores are provided by internal pores of the adsorbent particles. Typically, the odor-causing organisms are bacteria, but other odor-causing organisms may also be inhibited or operatively affected by the technique of the invention. The odor-causing organism may be mono-cellular or multi-cellular, and may comprise flora and/or fauna. The organisms may include bacteria, amoeba, fungi, yeast, rotifers or the like, as well as combinations thereof.

To provide the desired control of malodor, the target pores are configured with a selected pore-size. In a particular feature, the target pores can be configured to provide a pore-size of 1 nanometer (1 nm) or less. In another feature, the target pores can be configured to provide a pore-size of not more than a maximum of about 200 nm. The size of the target pores can alternatively be not more than a maximum of about 350 nm, and can optionally be not more than a maximum of about 500 nm to provide improved performance. In other arrangements, the pore size can be up to a maximum of about 1 micrometer (μm). If the pore-size is too large, the odor-causing organisms can too readily access any water in the target pores, and can excessively retain their ability to cause malodor.

In another aspect of the method and system, the quantity of target pores can be configured to be at least a minimum of about 3% of the total quantity of particle pores. The total quantity of target pores can alternatively be at least about 10% of the total quantity of particle pores, and can optionally be at least about 25% of the total quantity of particle pores to provide improved performance. Still other arrangements can have a total quantity of target pores which is at least about 50% of the total quantity of particle pores. In a further aspect, the quantity of target pores has been configured to be up to a maximum of about 0.80%, or more, of the total quantity of particle pores. The quantity of target pores can alternatively be up to about 90% of the total quantity of particle pores, and can optionally be up to about 100% of the total quantity of particle pores to provide a desired combination of lower cost and high performance.

If the quantity of target pores is too small, the water may not be adequately isolated from the odor-causing organisms. As a result, the odor-causing organisms can too readily access any free water in the structure, and can excessively retain their ability to cause malodor.

The total quantity of particle pores and the percentage of target pores can be determined by employing mercury porosimetry testing. Data regarding pore size, pore volume and pore size distribution can be obtained from Micromeritics Instrument Corp., One Micromeritics Dr., Norcross, Ga. 30093 U.S.A. The testing can include Macro and Meso Volume/Size Distribution by Mercury Intrusion Porosimetry, Test No. 005-65000-31, and test samples can be run on Micromeritics Instrument Corp.'s AUTOPORE Mercury Porosimeter, Unit 750.

A significant quantity of water can be held in the total quantity of target pores. In a particular aspect, the target pores can provide for a holding capacity of at least a minimum of about 0.5 grams water. The target pores can alternatively provide for a holding capacity of at least about 1 g of water, and can optionally provide for a holding capacity of at least about 2 g of water. In other arrangements, the holding capacity can be up to 5 g of water, or more.

In a further aspect, the target pores can be provided by particles of odor-control material wherein the particles have a particle size of between about 1,000 microns (μm) to about 100 μm; and, more desirably, between about 850 μm to about 150 μm. It has been found that particles of adsorbent material having a size above about 1,000 μm are generally, readily perceptible to the wearer of any containment or carrier structure that supports the adsorbent materials of the present invention. It has also been found that particles of adsorbent material having a size below 100 μm are difficult to contain within any containment mechanism that readily allows liquids to penetrate through the containment mechanism to the adsorbent materials. It should be understood that the particles of adsorbent material falling within the range identified herein may comprise individual porous particles, or may be agglomerated particles which each agglomerated particle comprising a plurality of smaller particles composed of one or more types of adsorbent materials.

In still another aspect, the method or system of the invention can include a configuring of the particles of adsorbent material to incorporate an operative quantity of odor-treatment material; and the odor-treatment material can be configured to cooperate with the viscous body-liquid to thereby generate a fragrance or other odor or that is relatively pleasing, or is otherwise, substantially non-objectionable.

A particular feature of the invention, the odor-treatment material can include milk protein, milk solids, dried milk or the like, as well as combinations thereof.

In another feature, the odor-treatment material can be carried or otherwise incorporated into the pores provided by the particulate material. A desired arrangement can incorporated the odor-treatment material within internal pores of the particulate material. The pores that are configured to carry the odor-treatment material can have a selected pore-size, and in a particular feature, the carrier pores can have a pore-size which is not more than about 1 μm. Pores above this size can be “loaded” with, or otherwise contain therein, at least one molecule of a selected material, such as the desired odor-treatment material. The molecule or molecules can then be released when the adsorbent material is exposed to water or other selected liquid.

The particulate material in the odor-control system may also include an absorbent material. The absorbent particulate material can, for example, include cellulose granules or bundles (“nits”), superabsorbent particles, coated superabsorbent particles, or the like as well as combinations thereof. For example, the particulate, absorbent material can be a FAVOR 880 polyacrylate superabsorbent material available from Stockhausen, a business having offices located in Greensboro, North Carolina, U.S.A.

It should be noted that the present invention is not limited to the use of only one of the adsorbent materials or other particulate materials disclosed herein, but can also include mixtures or other combinations of two or more materials. As previously indicated, the odor-control material is in particle form; consequently, use of the phrase “particulate material” throughout the specification and claims includes a quantity having one or more individual particles of material, or a quantity having agglomerations which include two or more particles of the same or different materials.

The odor-adsorbent or other odor-control particulate materials employed with the present invention can be appropriately combined with an operative carrier structure. In a particular aspect, the particulate material 28 can be held or otherwise carried by a suitable containment means or mechanism. Any means or mechanism which is capable of holding or otherwise carrying the described adsorbent materials, and is further capable of being located in a disposable absorbent article, can be employed in the present invention. Many such containment means or mechanisms are known to one skilled in the art. For example, the carrier structure may include a fibrous matrix such as an airlaid or wet laid web of cellulosic fibers, a meltblown web of synthetic polymeric fibers, a spunbonded web of synthetic polymeric fibers, a coformed matrix comprising cellulosic fibers and fibers formed from a synthetic polymeric material, an airlaid heat-fused web of synthetic polymeric material, an open-celled foam and the like, as well as combinations thereof.

In particular arrangements, the carrier structure can include a containment means or mechanism having at least two layers of material which are operatively joined together to form at least one pocket region or compartment region containing adsorbent material. Optionally, the carrier structure can be configured to form a plurality of two or more pocket regions. In the various arrangements of the pockets, at least one of the layers of containment material should be liquid-pervious. The second layer of material may be liquid-pervious or substantially liquid-impervious, as desired. Each layer of containment material may, for example, be a cloth-like woven or nonwoven fabric, a closed or open-celled foam, a perforated film, an elastomeric material, fibrous webs of material or the like, as well as combinations thereof. When the containment structure or mechanism includes one or more layers of material, the employed combination of material layers should provide a resulting pore structure that is small enough or tortuous enough to provide a containment structure that can operatively confine or otherwise hold at least a majority of the adsorbent material within the containment structure.

Alternatively, the carrier structure may be configured to provide a support means or mechanism. For example, a support structure can include a fiber, a woven or nonwoven fabric, a polymeric film or the like, as well as combinations thereof. The adsorbent material may be adhered or otherwise attached to one or both sides of the support structure, and the support structure may be configured to be liquid-pervious or substantially liquid-impervious, as desired.

The adsorbent material, and in desired arrangements, the odor-adsorbent material, can be present in the containment means or mechanism in an amount of from about 10 to about 100 weight percent (wt %); alternatively, about 20 to about 100 wt %; alternatively, about 30 to about 100 wt %; alternatively, about 40 to about 100 wt %; alternatively, about 50 to about 100 wt %; alternatively, about 60 to about 100 wt %; alternatively, about 70 to about 100 wt %; alternatively, about 80 to about 100 wt %; and alternatively, about 90 to about 100 wt % based on the total weight of the adsorbent and/or other material that has been operatively combined with the carrier structure.

In particular arrangements of the present invention, the article can provide another carrier structure which includes two or more, separately provided layers of material which are joined to form an operative pocket region configured to contain the adsorbent or other particulate material. One or more of the layers can be suitably formed from any material capable of containing the adsorbent or other particulate material, and the material can include woven and nonwoven materials such as fabric materials which include airlaid fibers, wet laid fibers, meltblown fibers, spunbonded fibers, coformed fibers, binder fibers (such as bicomponent fibers) and the like, as well as combinations thereof. The layers of material can be joined to form a pocket by a suitable securement mechanism, such as heat fusion, sonic bonding, adhesives (such as water-soluble or water-sensitive adhesives, latex adhesives, hot melt adhesives, or solvent-based adhesives) and the like, as well as combinations thereof. Clearly, any of a wide variety of materials may be employed to form the two layers, and any of a wide variety of securement techniques may be employed to join the two layers together to form the pocket. The adsorbent or other particulate material can be present in each pocket in an amount within in the range of about 10 to about 100 wt %; alternatively, about 20 to about 100 wt %; alternatively, about 30 to about 100 wt %; alternatively, about 40 to about 100 wt %; alternatively, about 50 to about 100 wt %; alternatively, about 60 to about 100 wt %; alternatively, about 70 to about 100 wt %/o; alternatively, about 80 to about 100 wt %; and alternatively, about 90 to about 100 wt % based on total weight of the adsorbent or other particulate material present in the pocket. In addition to the adsorbent or other particulate material, the pocket may contain a fibrous material or other filler material that does not unacceptably affect the properties of the adsorbent or other particulate material.

In another aspect, the carrier structure or mechanism of the article can include a matrix of fibers, and the adsorbent or other particulate material can be mixed with the fibers of the matrix. The adsorbent or other particulate material can be present in the mixture of fibers and adsorbent material in an amount of from about 20 to about 95 weight percent; alternatively, about 30 to about 85 weight percent; and alternatively, about 50 to about 75 weight percent based on total mixture weight.

Any fibers capable of containing an adsorbent or other particulate material and of forming a composite when in combination with the adsorbent or other particulate material are believed suitable for use in the present invention. It is often preferred that the fibers are hydrophilic. As used herein, a fiber or other material can be considered to be “hydrophilic” when it possesses a contact angle of water in air of less than 90 degrees. For the purposes of the present disclosure, contact angle measurements can be determined as set forth by Good and Stromberg in “Surface and Colloid Science”, Vol. 11 (Plenum Press, 1979).

Fibers suitable for use in the present invention include cellulosic fibers such as wood pulp fluff, cotton, cotton linters, rayon, cellulose acetate, and the like, as well as synthetic polymeric fibers. The synthetic polymeric fibers may be formed from inherently hydrophilic polymeric materials or may be formed from inherently hydrophobic polymeric materials (water in air contact angle of greater than 90 degrees), which fibers are then treated to render at least the outer surface of the fibers hydrophilic. For example, hydrophilic fibers may be formed from an intrinsically hydrophilic polymer such as a block copolymer of nylon, e.g., nylon-6, and a polyethylene oxide diamine. Such block copolymers are commercially available from Allied-Signal Inc. under the trade designation HYDROFIL. Alternatively, the fibers may be formed from an intrinsically hydrophobic polymer such as a polyolefin or polyester which has been surface modified to provide a generally non-fugitive hydrophilic surface. Such surface modified polyethylene is commercially available from the Dow Chemical Company under the trade designation ASPUN wettable polyethylene.

When the hydrophilic fibers are formed by applying a hydrophilic surface treatment to a generally hydrophobic polymer, it may be desirable to employ a generally non-fugitive surface treatment in order to obtain the desired performance.

The synthetic polymeric fibers suitable for use in the present invention may be formed by a melt-extrusion process wherein fibers of a polymeric material are extruded and attenuated to produce fibers having a desired diameter. Alternatively, the fibers may be formed through a spinning process. It is believed that any fiber-producing process known to one skilled in the art can be suitable for use in the present invention.

Fibers suitable for use in the present invention can generally have a length of at least about 1 millimeter. The fibers may have a maximum length approaching infinity. That is to say, the fibers may be essentially continuous such as those fibers formed through a meltblowing process under certain conditions known to one skilled in the art.

Reference to a mixture of fibers and adsorbent or other particulate material is intended to refer to a situation in which the adsorbent or other particulate material is in direct contact with the fibers or is not substantially prevented from migrating into contact with the fibers. Thus, for example, in a multi-layered adsorbent core in which the first layer comprises an airlaid mixture of wood pulp fluff and adsorbent or other particulate material and the second layer comprises only airlaid fluff, only the first layer is considered a mixture of fibers and adsorbent or other particulate material provided, however, that a significant dry migration of the adsorbent or other particulate material between the two layers is substantially prevented. Mechanisms and techniques for preventing such migration are known, and include separating the layers by a tissue wrap sheet, high density fiber layer or similar mechanisms or techniques to prevent a substantial dry migration of the adsorbent or other particulate material between the two layers. The mixture of adsorbent or other particulate material and fibers may be relatively homogenous or relatively non-homogeneous. In the case of a non-homogeneous mixture, the adsorbent or other particulate material may be arranged in a gradient or may be layered with the fibers.

When the containment mechanism or other carrier structure comprises a mixture of fibers and adsorbent or other particulate material, the mixture of fibers and particulate material may be formed in a variety of ways. For example, the mixture may be formed by airlaying or wet-laying the fibers and particulate material, according to processes known in the art, to form batts of the mixture. Airlaying the mixture of fibers and particulate material is intended to encompass both the situation wherein preformed fibers are airlaid with the adsorbent material as well as the situation in which the particulate material is mixed with the fibers as the fibers are being formed, such as through a meltblowing process.

The adsorbent or other particulate materials of the present invention are particularly suitable for use in disposable absorbent articles. In general, the particulate materials may be incorporated into conventional absorbent structures by employing well known techniques. For example, the particulate materials can be incorporated in laminates, in relatively high density cores (i.e., compacted cores, calendered cores, densified cores, etc.), or in relatively low density cores (i.e., not compacted, for example, air-laid cores). The particulate materials of the present invention, however, can provide certain advantages over conventional adsorbent or other particulate materials. In general, when compared to conventional particulate materials, the particulate materials of the present invention demonstrate an improved efficacy in the handling of complex liquids. In particular, the particulate materials of the present invention demonstrate an improved efficacy in the handling of menses. Additionally, the various aspects and configurations of the present invention can provide a more effective control of malodor. As a result, product developers can have the ability to either complement the absorbent systems typically employed in disposable absorbent articles with the various arrangements and configurations of the present invention, or replace certain absorbent systems with the various arrangements and configurations of the present invention.

With reference again to FIGS. 1, 2 and 2A, a representative tampon article can incorporate the odor-control system of the invention. As representatively shown in FIG. 2, odor-control particulate material 28 can be distributed through at least a selected central portion of an interior volume of the article. As representatively shown in FIG. 2A, the particulate material 28 can be distributed through a generally annular-shaped part of the interior volume of the article. In a particular configuration, the article can include an absorbent retention portion 42, and the particulate material of the odor-control system can be distributed in at least one layer region that extends completely or partially around a circumferential dimension of the article. Additionally, the one or more layer regions of the odor-control system can extend completely or partially along a longitudinal, length-wise dimension of the article. In the representatively shown arrangement, the odor-control system has its quantity of particulate material 28 located substantially subjacent a flexible, containment layer or other type of carrier layer 24, and located radially outboard from a cooperating retention portion 42. The retention portion is positioned relatively inboard from the particulate material 28, and toward a generally central region, as observed along the representatively shown, lateral cross-section of the article.

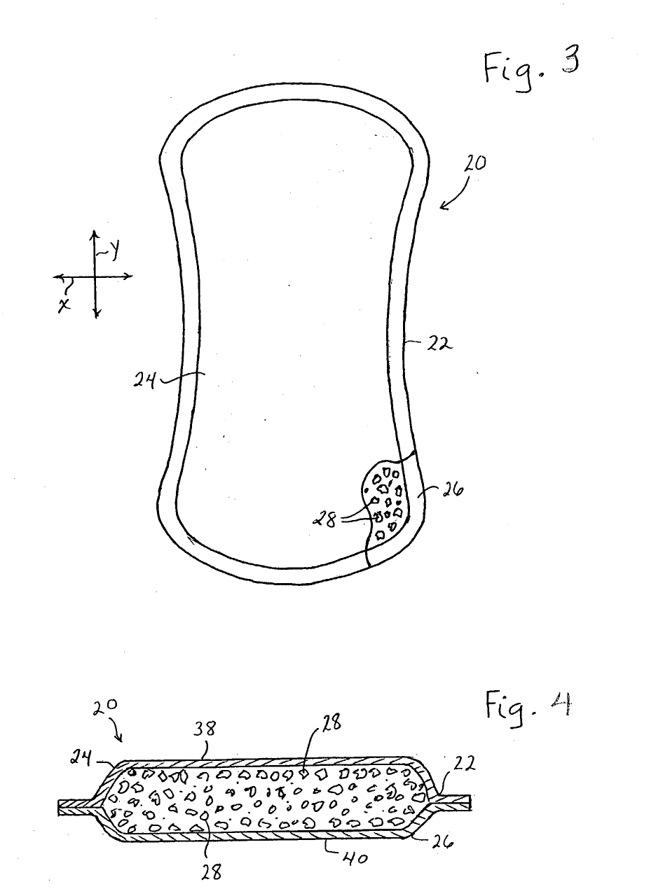

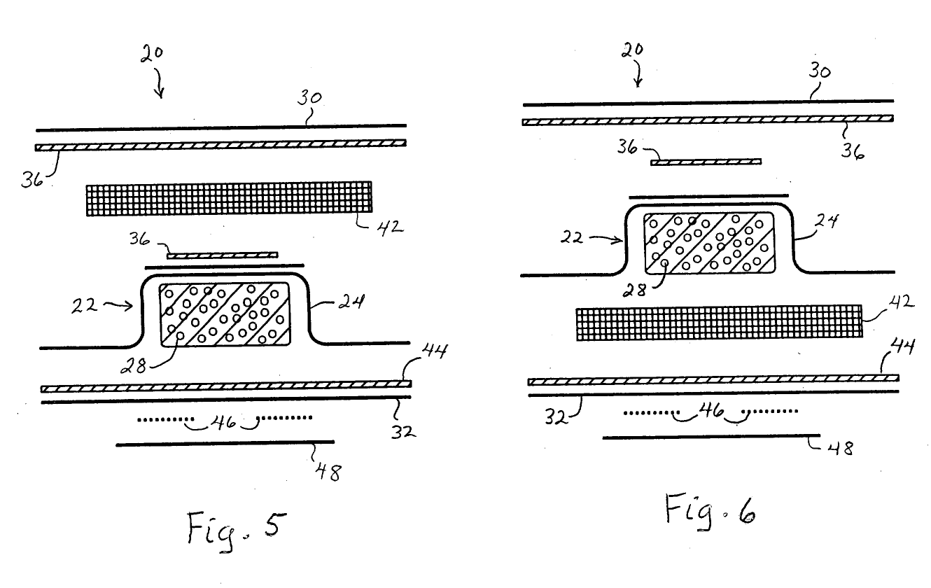

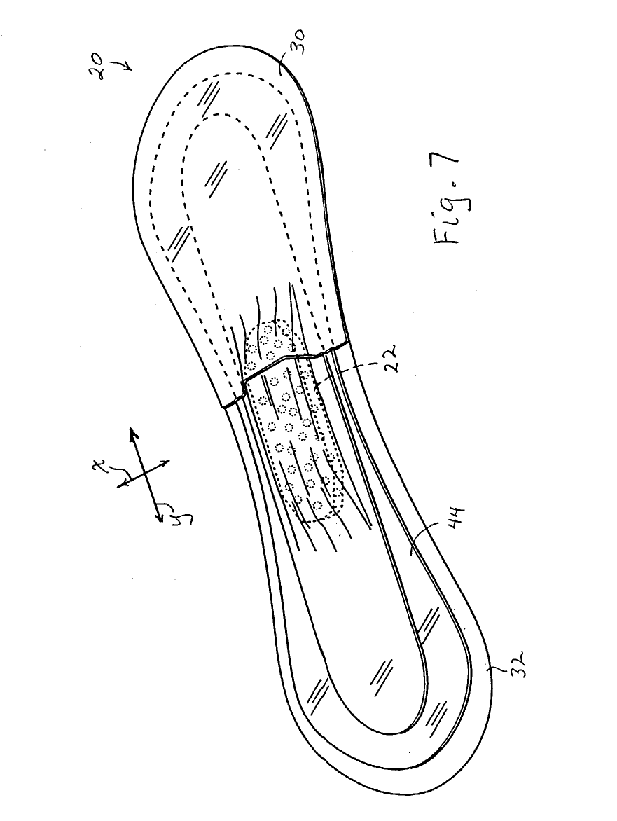

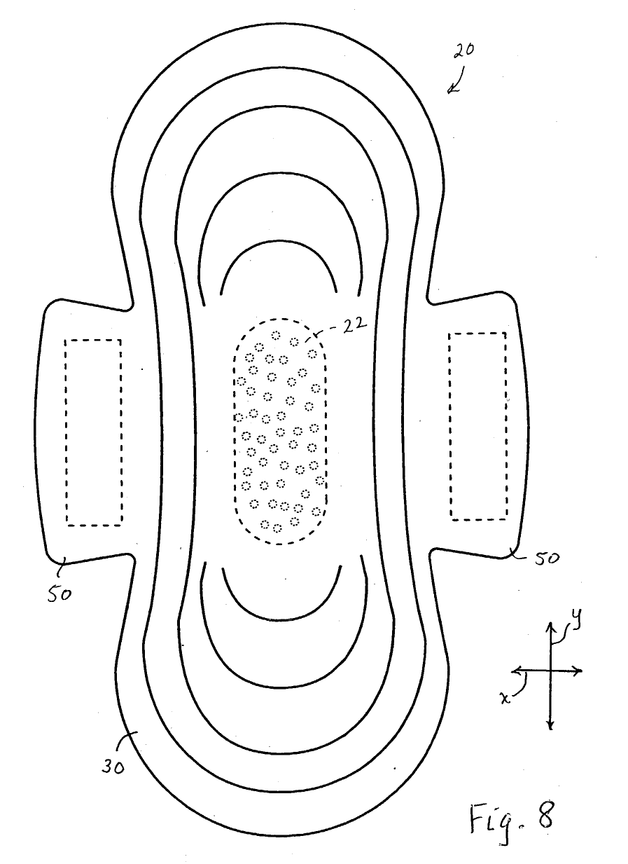

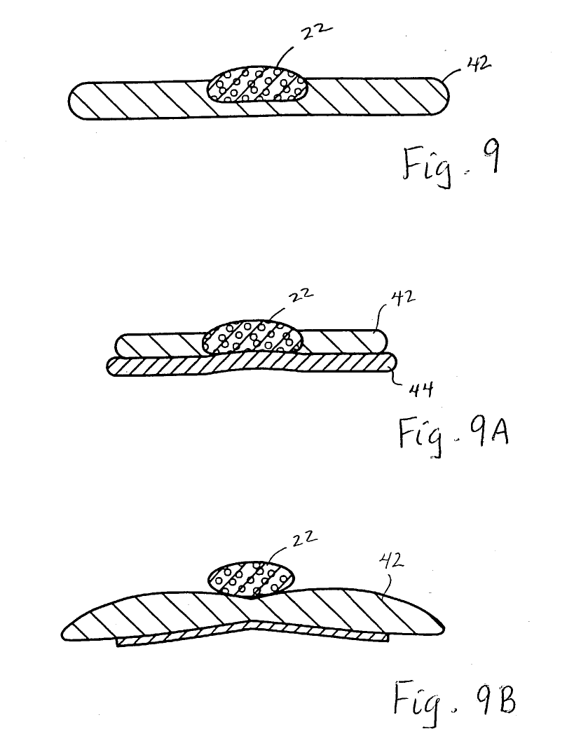

In a further aspect, the article and odor-control system can include a containment system or mechanism having a plurality of carrier layers, and the odor-control system or mechanism may comprise a laminate of at least two carrier/containment layers of material between which the adsorbent material is located and contained. In the representative arrangement illustrated in FIGS. 3 and 4, for example, the odor-control system 22 can include a first layer and a cooperating second layer. In a particular configuration, a liquid-pervious or liquid-permeable first layer 24 can be configured to provide a bodyside layer of the containment structure, and a second layer 26 can be configured to provide an outward, garment-side layer of the containment structure. The material of the second layer 26 may be the same as, similar to, or different than the material of the first layer 24. Additionally, the second layer 26 may be liquid-permeable or operatively liquid-impermeable, as desired.

In the odor-control system, each layer of containment material may, for example, be a cloth-like woven or nonwoven fabric, a closed or open-celled foam, a perforated film, an elastomeric material, fibrous webs of material or the like, as well as combinations thereof. When the containment structure or mechanism includes one or more layers of material, the employed combination of material layers should provide a resulting pore structure that is small enough or tortuous enough to provide a containment structure that can operatively confine or otherwise hold at least a majority of the adsorbent material within the containment structure.

The absorbent article 20 can further include a cover layer 30, a backsheet layer 32, and an absorbent core or retention portion 42 which is interposed between the cover layer and backsheet layer.

With reference to FIGS. 5 and 6, a representative absorbent article 20 can include an odor-control system 22, and a separately provided absorbent retention portion 42. Additionally, the representatively shown article can include a liquid-permeable cover layer 30, and an operatively liquid-impermeable baffle or backsheet layer 32. In the example of the shown arrangement, the flexible, carrier/containment layer 24 of the odor-control system 22 can maintain the position of the quantity of odor-control particulate material 28, and the baffle member can provide an operative garment-side layer of the odor-control system. The article can further include one or more other components, such as one or more distribution layers 36, and/or one or more article-shaping layers 44. The article-shaping layers may be configured to provide an absorbent component which can be a part of, or can otherwise cooperate with the retention portion. For example, the article can include a resilient, shaping-layer 44 which can be positioned relatively outward from, and immediately adjacent or otherwise operatively proximate a garment-facing surface of the quantity of odor-control particulate material. The shaping layer can be particularly desirable when the article is configured to be a feminine care article, such as a feminine care pad or liner. A garment-attaching adhesive 46 may be applied to a garment-side surface of the baffle layer, and a release sheet may be superposed over the garment adhesive. Typically, the release sheet is removed immediately prior to placing the article into use. As illustrated in FIG. 5, the retention portion 42 may be located on a bodyside of the odor-control system 22. As illustrated in FIG. 6, the retention portion can alternatively be located on a garment-side of the odor-control system.

The bodyside, cover layer 30 can be provided by any material that is operatively liquid-permeable, and may be a composite material. In a particular arrangement, the cover layer can be configured to provide at least a portion of the desired containment structure for the particulate material 28. The cover layer 30 can provide comfort and conformability, and can function to direct bodily exudates away from the body and toward the retention portion 42. In a desired feature, the cover layer 30 can be configured to retain little or no liquid in its structure, and can be configured to provide a relatively comfortable and non-irritating surface next to the body-tissues of a female wearer. The cover layer 30 can be constructed of any material which is also easily penetrated by bodily fluids that contact the surface of the cover layer. For example, the cover layer can include a woven fabric, a nonwoven fabric, a polymeric film that has been configured to be operatively liquid-permeable, or the like, as well as combinations thereof. Examples of suitable materials for constructing the cover layer can include rayon, bonded carded webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

An example of a suitable cover layer material would include a bonded-carded-web composed of polypropylene and polyethylene, such as has been used as a cover stock for KOTEX brand pantiliners, and has been obtainable from Vliesstoffwerk Christian Heinrich Sandier GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. The liquid-permeable cover layer 30 can also contain a plurality of apertures (not shown) formed therein which are intended to increase the rate at which bodily liquids can move through the thickness of the cover layer and penetrate into the other components of the article (e.g. the retention portion 42).

The cover layer 30 may also include a physiologically hydrous cover material. As used herein, the term “physiologically hydrous” is intended to connote a cover material which can maintain a suitably moist interface between the absorbent article 20 and any contacting body-tissues of the wearer that are ordinarily moist. For example, such moist-tissue regions are present in the vulvovaginal area of the female anatomy. The physiologically hydrous cover material can provide a desired level of comfort when disposed within the selected, moist-tissue environment of the wearer, keeping in mind as well the self-evident factor that the absorbent article may be receiving bodily liquids that may be migrating from the wearer to the article. Thus, while not “hydrous” in the classic sense, inasmuch as the cover layer will be substantially dry prior to use on the wearer, the cover layer 30 can maintain, or at least can avoid excessive interference with, a hydration level or balance that is desired within the ordinarily-moist body tissue.

The cover layer 30 can also have at least a portion of its bodyside surface treated with a surfactant to render the cover more hydrophilic. The surfactant can permit arriving bodily liquids to more readily penetrate the cover layer. The surfactant may also diminish the likelihood that the arriving bodily fluids, such as menstrual fluid, will flow off the cover layer rather than penetrate through the cover layer into other components of the article (e.g. into the retention portion). In a particular configuration, the surfactant can be substantially evenly distributed across at least a portion of the upper, bodyside surface of the cover layer 30 that overlays the upper, bodyside surface of the absorbent.

The cover layer 30 may be maintained in secured relation with the retention portion 42 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover layer 30 typically extends over the upper, bodyside surface of the retention portion, but can alternatively extend around the article to partially or entirely, surround or enclose the retention portion. Alternatively, the cover layer 30 and the baffle layer 32 can have peripheral margins which extend outwardly beyond the terminal, peripheral edges of the retention portion 42, and the extending margins can be operatively joined together to partially or entirely, surround or enclose the retention portion.

The baffle may, for example, include a polymeric film, a woven fabric, a nonwoven fabric or the like, as well as combinations or composites thereof. For example, the baffle may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the baffle 32 can operatively permit a sufficient passage of air and moisture vapor out of the article, particularly out of an absorbent (e.g. storage or retention portion 42) while blocking the passage of bodily liquids. An example of a suitable baffle material can include a breathable, microporous film, such as a HANJIN Breathable Baffle available from Hanjin Printing, Hanjin P&C Company Limited, a business having offices located in Sahvon-li.Jungan-mvu.Kongiu-City, Chung cheong nam-do, Republic of South Korea. The baffle material is a breathable film, which is white in color, dimple embossed, and contains, calcium carbonate, TiO₂, and polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable baffle material can include a closed-cell polyolefin foam. For example, a closed-cell polyethylene foam may be employed. Still another example of a baffle material would be a material that is similar to a polyethylene film which is used on commercially sold KOTEX brand pantiliners, and is obtainable from Pliant Corporation, a business having offices located in Schaumburg, Illinois, USA.

The retention portion 42 is configured to hold and store the liquids that are directed into the article. The retention portion 42 may include any operative absorbent material, such as cellulosic materials, other absorbent natural-materials, absorbent synthetic-materials, superabsorbent materials and the like, as well as combinations thereof. In a desired feature, the retention portion 42 can exhibit a menses-retention capacity that is within the range of about 1-35 gram menses-simulant per gram of retention material. In another feature, the retention portion 42 exhibits a total retention capacity that can be up to about 100 gram menses-simulant. In a further feature, the retention portion 42 can exhibit a urine-retention capacity that is within the range of about 2-50 gram of synthetic urine (0.9 wt % saline) per gram of retention material (g/g saline). The total holding capacity of the retention portion can be up to about 100 grams of synthetic urine or more. To provide the desired holding capacity for menses and/or synthetic urine, the retention portion may include superabsorbent material.

The retention capacity can be determined by employing the Method for Determining Retention Capacity that is identified in TESTING section of the present disclosure

In the various configurations of the invention, the desired substantially liquid-impermeable or substantially liquid-impervious material has a high-resistance and limited permeability to aqueous liquid, and can have a construction which is capable of supporting a hydrohead of at least about 45 cm of water substantially without leakage therethrough. A suitable technique for determining the resistance of a material to liquid penetration is Federal Test Method Standard FTMS 191 Article 5514, dated Dec. 31, 1968, or a substantially equivalent procedure.

With reference to FIGS. 7 and 8 the odor-control system 22 may be limited to extend along only a selected portion of the length (y) and/or width (x) dimensions of the article. Alternatively, the odor-control system can be configured to extend along a total length dimension of the article, and/or may be configured to extend along a total width dimension of the article, as desired. As illustrated in FIG. 8, the article may further include extending tabs, which can be folded around the edges of an undergarment in the crotch region of the wearer to help maintain a desired positioning of the article.

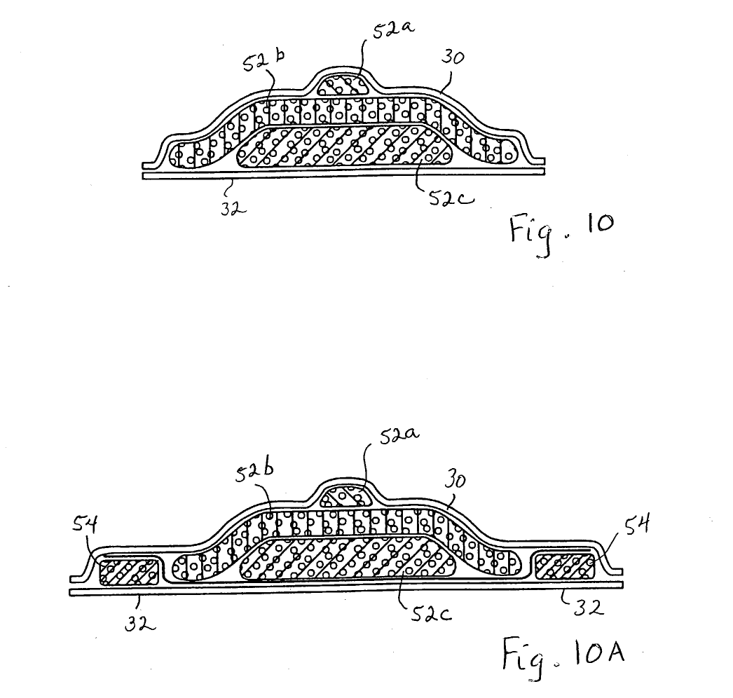

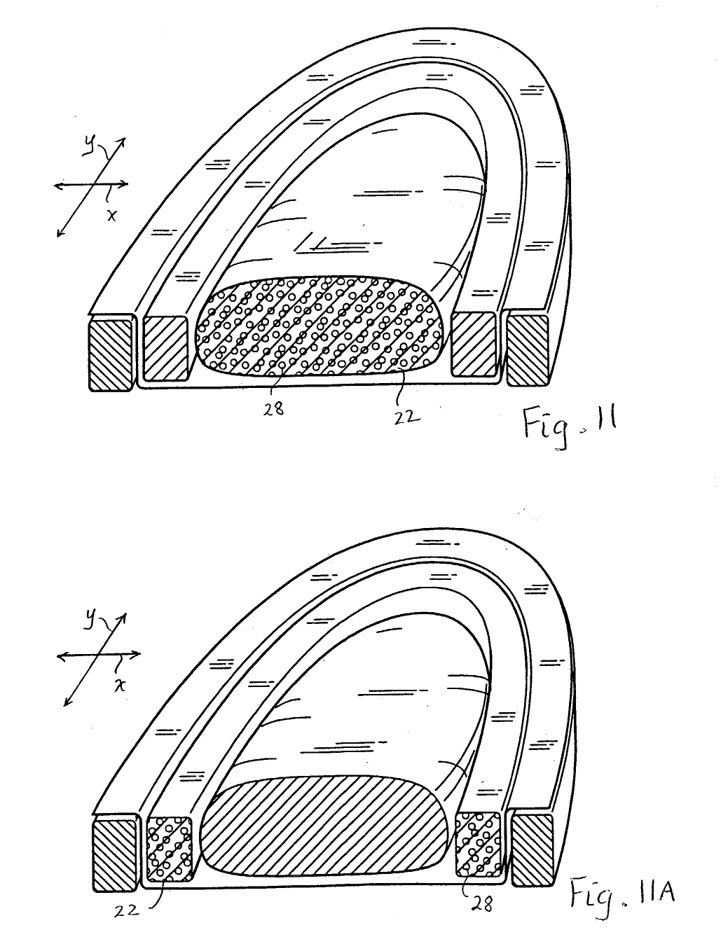

With reference to FIGS. 9 through 9B, the odor-control system 22 can be configured to cooperate with the retention portion 42 in various alternative arrangements. The odor-control system can, for example, be partially sunken into a z-directional thickness of the retention portion 42 (e.g. FIG. 9). The odor-control system 22 can optionally be substantially totally sunken into the z-directional thickness of a selected retention portion 42 (e.g. FIG. 9A). In other arrangements, the odor-control system may be entirely superposed over, or entirely positioned under the retention portion 42 (e.g. FIG. 9B), as desired.

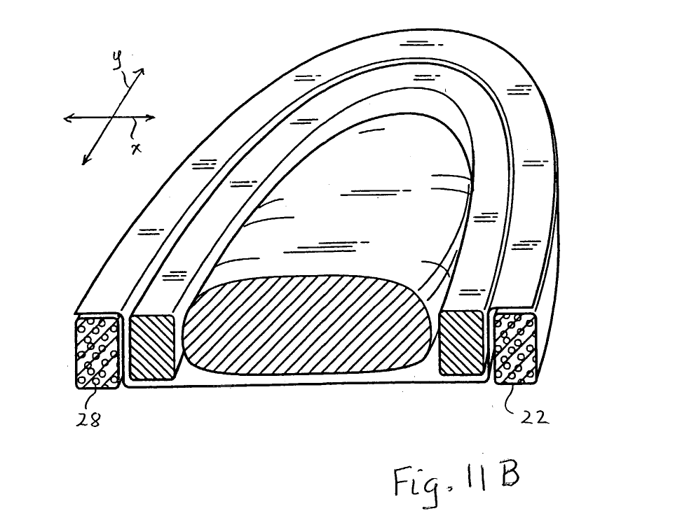

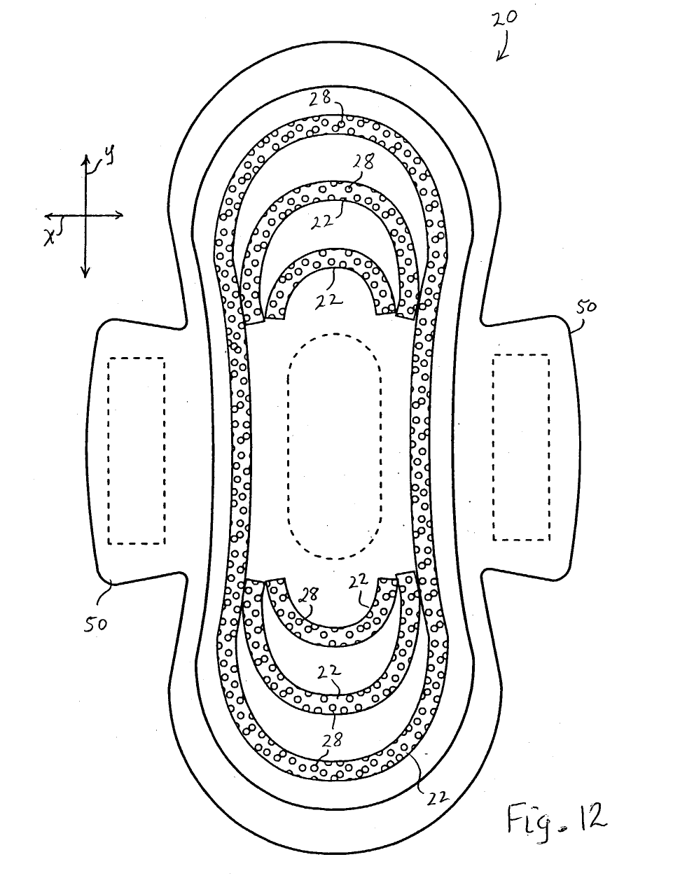

As representatively shown in FIGS. 10 and 10A, the article can include a plurality of discrete layer regions or strata. The odor-control system 22 may be incorporated into two or more of the layer regions, and the layer regions that provide the odor-control system may or may not be in direct contact with one another. With reference to FIG. 10, for example, the article may include three, substantially superposed layer regions 52a, 52b, 52c. The layer regions can extend generally parallel to each other, and may be of different sizes or may be approximately the same size. The particulate material of the odor-control system may be incorporated into any one of the layer regions, or may be incorporated into all of the layer regions. Alternatively, the particulate material may be incorporated into any other desired combination of two or more of the layer regions. With reference to FIG. 10A, the article can include an array of laterally outboard edge components 54, and the odor-control system may be selectively incorporated into the edge components With reference to FIGS. 11 and 11A, a representative article can have an array of article components that are distributed along x-y dimensions of the article. The individual components of the array may be absorbent or non-absorbent, as desired. Additionally, the individual components can extend generally continuously along the article, but may be configured to extend discontinuously along the article. The article components may have the form of circular or non-circular rings, and immediately adjacent rings may or may not be arranged to extend generally parallel to each other. The odor-control system 22 can, for example, be a component which is arranged at a generally medial position with respect to the array of absorbent components (e.g. FIG. 11). Alternatively, the odor-control system can be arranged in an intermediate position with respect to the array of absorbent components that are distributed along x-y dimensions of the article (e.g. FIG. 11A). Optionally, the odor-control system can be located at a selected outboard position with respect to the array of absorbent components of the article (e.g. FIG. 11B).

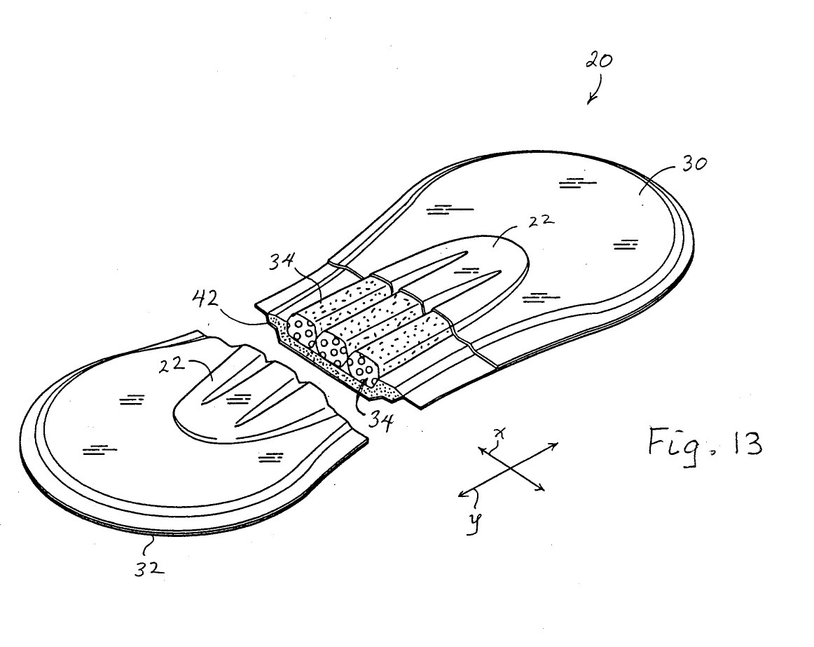

As representatively shown in FIG. 12, an article which incorporates the invention can include an odor-control system 22 that is configured into any selected combination of individual components, and the components can be distributed and arranged in any operative array of odor-control components. Each individual odor-control component can have a discontinuous extent, or a generally continuous extent, and the various odor-control components can be distributed along any of the various dimensions of the article.

FIG. 13 is a representative, perspective view of a partially sectioned article having an odor-control system 22 which is configured with a varying contour. In a particular arrangement, the odor-control system can be configured to provide a regular or irregular array of individual, discrete pocket regions 34. In the illustrated example, the pocket regions are individually formed along a width dimension of the article. The pocket regions may alternatively be individually formed along a length dimension of the article, and may optionally be individually formed along both the width and length dimensions of the article. The illustrated example has pocket regions that are immediately adjacent each other. Alternatively, the pocket regions may be spaced apart by any operative distance or combination of distances. Additionally, the pocket regions may be configured to have any operative shape and volume.

In a particular arrangement, the containment means or mechanism can have at least two layers of material which are operatively joined together to form at least one pocket region or compartment region that contains the adsorbent material. Optionally, the article and odor-control system can be configured to form a plurality of two or more pocket regions 34 (e.g. FIG. 13). In the various arrangements of the pockets, at least one of the layers of containment material can have an operative level of liquid-permeability, and the liquid-permeable layer may or may not be positioned on a body-side of the odor-control system. The second layer of material may have a selected level liquid-permeability or may be substantially liquid-impervious, as desired.

It should be readily appreciated that in the various configurations of the invention, the carrier structure can provide at least a portion of a personal care article. The personal care article can, for example, be a bed pad, an infant diaper, a child's training pant, an adult incontinence product, or the like. In a desired configuration the personal care article can be a feminine care article, such as a garment liner, a sanitary pad, a tampon, a miniform or interlabial device, or the like.

To determine various parameters that are set forth in the present disclosure, suitable test equipment and procedures are described in detail in PCT Publication WO 00/62826 published Oct. 26, 2000 and entitled ADSORBENTS FOR USE IN HANDLING COMPLEX FLUIDS by William G. Reeves et al. The entire disclosure of this document is incorporated herein by reference in a manner that is consistent herewith. The test methods described in this document include: Intake Rate and Rewet Test Method; Method for Determining Retention Capacity; Capillary Tension Test Method; Gel Bed Permeability Test Method.

The following Examples describe various configurations of the invention, and are presented to provide a more detailed understanding of the invention. Other arrangements within the scope of the claims will be apparent to one skilled in the art from consideration of the present disclosure.

### EXAMPLE 1

In this example, 10 milliliter (mL) volumes of ZEOFREE 5175 were insulted with from 2 to 10 mL of menstrual simulant and allowed to stand. The ZEOFREE 5175 was a commercially available, granulated silicate material obtained from the J. M. Huber Corporation, a business having offices located in Havre de Grace, Maryland, U.S.A. No unpleasant odors developed, even when the samples were allowed to stand overnight at room temperature. A similar volume of fluffed pulp, insulted with a like amount of menstrual simulant, produced a noticeably unpleasant odor within two hours and was difficult to approach due to the extreme unpleasantness of the odors when left overnight. Similar odor suppression was observed with other high surface area materials, including Cab-O-Sil and Sipernat 50S.

### EXAMPLE 2

In this example, ZEOFREE 5175B was coated with 20% milk solids using a conventional fluidized-bed coater. When a 10 ml volume of this material was insulted with 6 ml of menses simulant, the material not only suppressed any malodor production for a period exceeding 24 hours, but also unexpectedly produced a pleasant odor. Other wetting agents, such as TWEEN 20, SYNTHRAPOL KB, and AEROSOL OT, did not interfere with the suppression of malodor when coated onto particles of adsorbent that met the requirements of the current invention, but did not help produce any unexpected odors, pleasant or otherwise.

The above-disclosed Examples are not intended to limit the scope of the present invention in any way. It is intended that the specification, together with the Examples, be considered exemplary only, with the scope and spirit of the invention being indicated by the claims which follow. Various modifications and other embodiments and uses of the disclosed superabsorbent-containing composites, apparent to those of ordinary skill in the art, are also considered to be within the scope of the present invention.

1. A method for reducing malodor, comprising:
a providing of an operative quantity of particles of adsorbent material with a carrier structure; and
a configuring of said carrier structure to contact at least one viscous, aqueous body-liquid during an intended use of the carrier structure;
said carrier structure having been configured to reduce malodor from the viscous body-liquid;
said particles of adsorbent material having been configured to include a total quantity of particle pores;
at least a significant portion of said particle pores having been configured to provide an operative quantity of target pores;
said target pores having been configured withdraw and hold water from said body-liquid; and
said target pores having been configured to hold the water in a manner that renders the water substantially inaccessible to odor-causing organisms.

2. A method as recited in claim 1, wherein said target pores have been configured to provide a pore-size of not more than about 1 μm.

3. A method as recited in claim 1, wherein said target pores have been configured to provide a pore-size of not more than about 500 nm.

4. A method as recited in claim 1, wherein said target pores have been configured to provide a pore-size of not more than about 350 nm.

5. A method as recited in claim 1, wherein said quantity of target pores has been configured to be at least about 3% of the total quantity of particle pores.

6. A method as recited in claim 1, wherein said quantity of target pores has been configured to be at least about 10% of the total quantity of particle pores.

7. A method as recited in claim 1, wherein said quantity of target pores has been configured to be at least about 25% of the total quantity of particle pores.

8. A method as recited in claim 1, wherein said odor-causing organisms include odor-causing bacteria.

9. A method for reducing malodor, comprising:
a providing of an operative quantity of particles of adsorbent material with a carrier structure;
said carrier structure having been configured to contact at least one viscous, aqueous body-liquid during an intended use of the carrier structure, and to reduce malodor from the viscous body-liquid;
said particles of adsorbent material having been configured to include a total quantity of particle pores;
at least a significant portion of said particle pores having been configured to provide an operative quantity of target pores;
said target pores having been configured to withdraw and hold water from said body-liquid; and
said target pores having been configured to hold the water in a manner that renders the water substantially inaccessible to odor-causing bacteria;
said target pores having been configured to provide a pore-size of not more than about 200 nm;
said quantity of target pores having been configured to be at least about 10% of the total quantity of particle pores.

10. A method for reducing malodor, comprising:
a providing of an operative quantity of particles of adsorbent material with a carrier structure; and a configuring of said carrier structure to reduce malodor from at least one viscous, aqueous body-liquid;
said particles of adsorbent material having been configured to include a total quantity of particle pores;
said particles of adsorbent material configured to include an operative quantity of odor-treatment material; and
said odor-treatment material having been configured to cooperate with the viscous body-liquid to thereby generate a substantially non-objectionable odor.

11. A method as recited in claim 10, wherein at least a significant portion of particle pores have been configured to provide a quantity of carrier pores having a pore-size of not more than about 1 μm.

12. A method as recited in claim 10, wherein at least a significant portion of particle pores have been configured to provide a quantity of target pores having a pore-size of not more than about 500 nm.

13. A method as recited in claim 10, wherein at least a significant portion of particle pores have been configured to provide a quantity of target pores having a pore-size of not more than about 350 nm.

14. A method as recited in claim 10, wherein at least a significant portion of particle pores have been configured to provide a quantity of target pores having a pore-size of not more than about 200 nm.

15. A method as recited in claim 10, wherein said quantity of target pores has been configured to withdraw and hold water from said body-liquid in a manner that renders the water substantially inaccessible to odor-causing organisms.

16. A method as recited in claim 10, wherein said odor-treatment material has been selected from the group consisting of milk solids, milk protein and dried milk.

17. A method as recited in claim 1, further including a configuring of said carrier structure to provide at least a portion of a personal care article.

18. A method as recited in claim 1, further including a configuring of said personal care article to include a cover layer, and a baffle layer which operatively joined to the cover layer; wherein said particles of adsorbent material are positioned between the cover layer and baffle layer.

19. A system for reducing malodor in a personal care article, said system comprising:
a personal care article configured to retain at least one viscous, aqueous body-liquid; and
at least one adsorbent particulate material disposed within the personal care article;
said at least one adsorbent particulate material having been configured to provide a total quantity of particle pores configured to withdraw and hold water from at least one viscous, aqueous body-liquid;
a substantial portion of said total quantity of particle pores having been configured to hold water in a manner that renders the water substantially inaccessible to odor-causing organisms; and
an operative quantity of such adsorbent particulate material having been disposed within the personal care article to operatively contact said at least one viscous, aqueous body-liquid during use such that water from the viscous, aqueous body-fluid is rendered substantially inaccessible to odor-causing organisms, to thereby reduce malodor from said personal care article.
